(19) United States
(12) Patent Application Publication
(10) Pub. No.: US 2004/0029294 A1
(43) Pub. Date: Feb. 12, 2004

(54) Method for determination of the origin and authenticity of materials with biological, biochemical and nutritional physiological relevance

(76) Inventors: Hanns-Ludwig Schmidt, Landshut (DE); Claudio Fuganti, Milan (IT)

Correspondence Address: MARTIN FLEIT, FLEIT KAIN GIBBONS GUTMAN & BONGINI P.L., 601 BRICKELL KEY DRIVE, MIAMI, FL, 33131, US

(21) Appl. No.: 10/459,414

(22) Filed: Jun. 10, 2003

Related U.S. Application Data
(63) Continuation of application No. PCT/EP01/15071, filed on Dec. 19, 2001.

(30) Foreign Application Priority Data
Dec. 21, 2000 (DE) 100 64 457.0
Jul. 26, 2001 (DE) 101 36 460.1

(51) Int. Cl. G01N 33/00; G01N 33/543

(52) U.S. Cl. 436/518; 436/127

(57) ABSTRACT

A method for determining the origin of substances with biological, biochemical and nutritional physiological relevance and distinguishing them from synthetic analogs using their relative positional oxygen or hydrogen isotope ratios (δ¹⁸O value and δ²H value), in which—based on the fact that for organic compounds there are at least two different primary or secondary sources for these elements, each with different isotope values, and the incorporation of these elements is accompanied by different, but characteristic effects—the positional δ¹⁸O values or δ²H values are individually determined in the substances to be tested and they are compared with the values expected from biosynthesis, where a correspondence or deviation allows the authenticity or origin or adulteration of the substance to be detected.

### RELATED APPLICATION

This application is a continuation of PCT International Application No. PCT/EP01/15071 filed Dec. 19, 2001, the contents of which are here incorporated by reference in their entirety.

### BACKGROUND OF THE INVENTION

1. Field of the Invention

This invention relates to a method for the determination of the global and positional oxygen and hydrogen isotope content of organic compounds for purposes of determining their origin and synthesis based on scientific findings, interpretations or predictions derived from the mechanisms of the corresponding (bio)syntheses.

2. Prior Art

Like all primary biological elements, oxygen and hydrogen occur in nature as mixtures of stable isotopes with “relative average frequencies” ¹⁶O=99.6337 Atom %, ¹⁷O=0.0374 Atom % and ¹⁸O=0.2039 Atom %, and ¹H=99.9822 Atom %, ²H=0.0145 Atom %. To characterize the isotope content of certain substances, the isotope ratio R is used for the most part; it is the ratio of the relative frequency of a heavy isotope to that of the principal isotope (for example, for ¹⁸O on average R=0.0020439, and for ²H is it 0.000145). Finally, precise data of positional and temporal deviations from the average value are given in the so-called δ values as the relative differences to the isotope ratio of an international standard. The standard for ¹⁸O and ²H is the “Vienna Standard Mean Ocean Water” (V-SMOW) of the IAEA in Vienna, with R=0.0020052, or 0.00015576, respectively. Relative to this, we have for any sample, for example,

δ¹⁸O value[0/00]_{v-SMOW}=[Rₛₐₘₚₗₑ/R_{v-SMOW}−1]×1000

To determine the global oxygen isotope ratio of a sample, it is decomposed by pyrolysis under exclusion of O₂ in a vacuum system, converting the organically bound oxygen to CO; H₂O is isotopically equilibrated with CO₂. The measurement gases are analyzed in an isotope ratio mass spectrometer, automatically in a comparison with the standard, for the ratio of the corresponding isotopomeric molecules, for example in the case of CO for the ratio of the masses ¹²C¹⁶O (M=28) and ¹²C¹⁷O (M=30). Frequently, pyrolysis isotope ratio mass spectrometry (P-IRMS) is coupled to the gas chromatographic separation of the compounds to be investigated (GC-P-IRMS). The required amount of substance is about 0.1 mg per compound and the error range of the measurement, including sample preparation, is 0.3%.

Positional isotope analyses (determination of isotope patterns) require the controlled decomposition of the organic compounds by appropriate chemical procedures and isotope ratio determination of the fragments; in this case about 20 mg of sample is needed in each case.

The determination of deuterium (²H) patterns in organic natural substances by nuclear magnetic resonance spectroscopy (NMR) has long been routine for detection of the sugar levels of wine and of fruit juices (Martin, G. J., Martin, M. L. (1988): The site-specific natural isotope fractionation-NMR method applied to the study of wines. In Linskens, H. F., Jackson, J. F. (eds.): Modern Methods of Plant Analysis, Vol. 6. Springer Verlag, pp.258-275; Martin, G. (1992): SNIF-NMR—A method for detecting a beet sugar additive in fruit juices. Flüssiges Obst. 59, pp. 477-485) and to determine the origin and authenticity of flavorings and fragrances (Martin, G. J., Hanneguelle, S., Remaud, G. (1990): Authentication of flavor and aroma by nuclear magnetic resonance and mass spectroscopy of the isotope ratio. Parums Cosmét. Arômes 94, 95-109) (FR-A-2517828; EP-A-0090901; U.S. Pat. No. 4,550,083). Assignment of origin and investigation of authenticity of unknown samples by means of this method are currently carried out nearly exclusively solely on an empirical basis, by comparing data with those of authentic reference material. The only example of causal interpretation of a deuterium pattern (to be sure, partially determined by mass spectrometry) is the connection of the relative ²H frequency in the carbonyl group of benzaldehyde (bitter almond oil) and its synthesis that is described (Butzenlechner et al., 1988).

In the synthesis of organic compounds, the isotope ratios of all bioelements undergo slight shifts because of thermodynamic or kinetic isotope effects, i.e., the different behavior of isotopomeric molecules in phase transitions or (bio)chemical reactions (i.e., changes of the of the ratios of isotopomeric molecules in a given population). For example, the different species of water (¹H¹H¹⁶O, ¹H²H¹⁶O, ¹H¹H¹⁸O, etc.) have different vapor pressures that in the case of evaporations and condensations lead to shifts of the ratios between the distillation receiver and the condensate.

Accordingly, the hydrogen and oxygen isotope content of the water in the leaves of plants, thus the isotope contents of natural substances synthesized from them, are primarily determined by seasonal and local climatic factors (precipitation, evaporation), and further through characteristic isotope discriminations in the course of biosynthesis due to the mechanisms and isotope effects of the reactions that are involved. On the other hand, the analysis of the global and positional isotope contents of natural substances, foods or their ingredients provided evidence about their geographic and biological origin as well as the contributing synthesis reactions and their conditions.

In the case of the carbon isotope ratios the interpretation and even the prediction of data today is largely scientifically based; in the case of hydrogen and oxygen it is limited to the above noted connections with climatic factors, but here the interpretation essentially depends on empirical data, i.e., on comparison with authentic standard substances and the formation of data banks. Approaches to systematic causal interpretations or predictions of frequencies or patterns of all bioelements in natural substances are found in earlier works by the inventor (Schmidt H. L., Kexel H., Butzenlechner M., Schwarz S., Gleixner G., Thimet S., Werner R. A., Gensler M. [1995]: Non-statistical isotope distributions in natural compounds: Mirror of their biosynthesis and key for their origin assignment. In Wada E., Yoneyama T., Minagawa M., Ando T., Fry B. D. (Eds.), Stable Isotopes in the biosphere. Kyoto University Press, Kyoto, 17-35);

(Schmidt H. L., Gleixner G.[1998]: Isotopic patterns in natural compounds. Origin and importance in authenticity analysis. In Schreier P., Herderich M., Humpf H. U., Schwab W. (Eds.). Natural Product Analysis, Chromatography—Spectroscopy—Biological Testing. Friedrich Vieweg & Sohn Verlagsgesellschaft mbH, Braunschweig, 271-280).

Up to now the following practical results in the determination of origin and authenticity in the case of foods and their ingredients were achieved on an empirical basis via global ¹⁸O analyses, often combined with the corresponding determinations by means of other elements: Determination of the origin of wine based on ¹⁸O analyses of water, [Roβmann A., Reniero F., Moussa I., Schmidt H. L., Versini G., Merle M. H. [1999]: Stable oxygen content of water EU data bank wines from Italy, France and Germany. Z. Lebensm.-Unters. Forsch. A208, 169-176]. Detection of the addition of water or sugar to fruit juices and differentiation between original juices and reconstituted fruit juice concentrates [Bricout, J., Merliot L. Fontes J.-C. [1973]: The composition of a stable isotope in the water of apple juice. Indust. Aliment. Agric. 90(1), 19-22; Rossmann A. Trimborn P. [1996]: The contents of stable oxygen isotopes in water of apple juice concentrate as a criterion for detecting sugar adulteration. Z. Lebensm. Unters. Forsch. 203, 277-282; Houeron G., Kelley S. D., Dennis M. J. [1999]: Determination of the oxygen-18/oxygen-16 isotope ratios of sugar, citric acid and water from single strength orange juice. Rapid Communications in Mass Spectrometry 13, 1257-1262]. Determination of the origin of butter (relevant in cases of subsidy fraud) [Roβmann A., Haberhauer G., Holzl S., Horn P., Pichlmayer F., Voerkelius S. [2000]: The potential multielement stable isotope analysis for the regional origin assignment of butter. Eur. Food. Res. Technol. 211, 32-40]; the procedure is also in principle valid for milk and other milk products as well as for meat. Determination of the origin of olive oil [Angerosa F., Breas O., Contento S., Guillon C., Reniero F., Sada E. [1999] Application of stable isotope ratio analysis to the characterization of the geographical origin of olive oils. J. Agric. Food Chem. 47, 1013-1017] or glycerol (attribution to plant, animal and synthetic sources, possible importance in adulteration of wine [Roβmann A., Schmidt H. L., Hermann A., Ristow R. [1998]: Multielement stable isotope ratio analysis of glycerol to determine its origin. Z. Lebensm.-Unters. Forsch. A 207, 19-30]). Differentiation between natural and synthetic flavorings, for example “furaneol,” (flavoring from strawberries) or vanilla.

In all of these cases only the global δ¹⁸ O values were determined, either the values of water itself or of substances whose δ¹⁸O value correlates with that of the water present in their creation. The assignment of origin essentially is based on the fact that the global δ¹⁸O value of the precipitation and ground water, thus, the water in the leaves and fruit, is connected with the geographic location of the area under cultivation. However, all values can be affected by local and seasonal factors (rain before the wine harvest, other local climatic effects); therefore, they can overlap considerably, so that in the case of legally relevant quality examinations they are mostly combined with other parameters.

Positional δ¹⁸O values, moreover, are determined by biochemical factors. The only results for positional δ¹⁸O values published up to now (Fronza G., Fuganti C., Pedrocchi-Fantoni G. Serra S., Zucchi G., Fauhl C., Guillou C., Reniero F. [1999]: Stable isotope characterization of raspberry ketone extracted from Taxus baccata and obtained by oxidation of the accompanying alcohol (betuligenol). J. Agric.Food Chem.47, 1150-1155), however, do not contain a systematic interpretation based on biosynthesis.

Similar considerations can be made with regard to the δ²H values. Reference is made to the measurement technique mentioned above, particularly as noted in the Martin et al. publications.

### SUMMARY OF THE INVENTION

As already noted, the use of all the previously mentioned tests for origin assignments is almost exclusively based on determinations of the δ¹⁸O value of water or organic substances isotopically correlated with water. With one exception these involve determinations of global values combined with comparison of the corresponding authentic reference substances. In contrast, this method invention is based on the inventors' recognition of biosynthetic relationships, interpretations and predictions of oxygen isotope contents and isotope patterns of all conceivable natural substances (and their synthetic analogs) for which the (bio)synthesis is known. In the case of hydrogen isotope patterns, this concerns above all aromatic compounds. Accordingly, based on the isotope contents of the primary sources of the oxygen in organic compounds (CO₂, H₂O, O₂), the mechanism of their (bio)syntheses and the related known and expected isotope effects (see scheme according to Schmidt et al., noted above), it is possible today to predict the δ¹⁸O of any natural substance; all of the currently available experimental data corroborate the corresponding predictions. Conversely, from the global δ¹⁸O value of a natural substance with many oxygen atoms, its δ¹⁸O values and thereby the origin of the organic compound can be derived on the basis of correlations to the leaf water of the plants that were found by the inventors (see FIG. 1, Scheme 1).

With regard to the ²H value, systematic evaluations of accessible ²H NMR data for natural substances from the group of the phenyl propane in a similar connection lead to the finding that here one can have a general systematization of the relative deuterium frequency in the aromatic ring that derives from the biosynthesis mainly with the frequency sequence p>o>m, while in the case of synthetic analogs, a statistical deuterium distribution is found as expected, see FIG. 4 (Scheme 4).

In addition, natural aromatic compounds, in connection with hydroxylation in p position, must experience a change of the relative positional ²H frequency because of the reaction mechanism (NIH shift, migration of the H atom [enriched in this connection] from the substituted position to the adjacent position).

The task of the invention accordingly is to make available new methods in order to

a) predict and determine the origin and authenticity of flavorings, foods and ingredients via the δ¹⁸O value without first producing data banks of corresponding comparison substances, and

b) predicting the suitability of test substances for reliable origin determination or other characterization of foods, flavorings or natural drugs as well as other questions, for example from the field of nutrition.

The foregoing is accomplished by the present invention by providing a method for determining the origin of substances with biological, biochemical and nutritional physiological relevance and distinguishing them from synthetic analogs using their relative positional oxygen or hydrogen isotope ratios (δ¹⁸O value and δ²H value) comprising the steps of determining—based on the fact that for organic compounds there are at least two different primary or secondary sources for these elements, each with different isotope values, and that the incorporation of these elements is accompanied by a different, but characteristic isotope effect—the positional δ¹⁸O values or δ²H values individually in the substances to be tested, comparing them with the values expected from the biosynthesis, and detecting where a correspondence or deviation makes it possible to determine the authenticity or origin or adulteration of the substance. In a further refinement of the method, includes the step of selecting from the the average value of all of the positional isotope values that occur in the substance to be tested, one of the said isotopes, measuring said isotope, comparing with the average δ¹⁸O value expected from the correlation to the starting substances, and making a determination concerning the authenticity or origin or adulteration of the substance. The method of the present invention also includes the step of determining, where in biological material the origin of the oxygen from leaf water (δ¹⁸O value +5 to −5 [%]_{V-SMOW}, 2-8 [%]_{V-SMOW} over that of the relevant ground water) or atmospheric O₂ (δ¹⁸O value+23.5 [%]_{V-SMOW}), the O atoms of which experience a characteristic isotope discrimination upon incorporation. The invention further contemplates the step of comparing, the positional hydrogen (δ²H) isotope value in aromatic compounds to be determined by ²H NMR with the relative ²H frequency p>o>m to be expected for natural substances arising by the shikimic acid pathway or for substances substituted in p position with OH, o>m or m>o, in each case according to biosynthesis. The method also contemplates that the substance to be tested, L-tyrosine is in the form of a metabolite or a component of a (macro)molecule or molecular aggregate. Also, the method includes the step of determining the plant or animal origin of tyrosine on the basis of the different origin of oxygen and/or hydrogen and the different isotope effects when they are incorporated into tyrosine.

The ¹⁸O isotope frequencies and patterns of natural substances and the corresponding synthetic analogs are derived on the basis of known (bio)syntheses, their mechanisms, the reaction conditions and isotope effects. On this basis, experimental data for unknown samples enables an interpretation of their origin and authenticity. In particular, the differentiation of L-tyrosine of plant or animal origin is initially intended as a basis for detection of the use of animal protein sources in the feeding of ruminants.

The positional determination of the δ¹⁸O values of L-tyrosine in order to detect the feeding of animal meals to ruminants, thus detection of circumvention of the corresponding prohibition, is proposed as a corresponding example. Tyrosine from purely animal biosynthesis (from phenylalanine) [theoretical δ¹⁸O value 5+2%] has to differ characteristically and unambiguously from that from plant sources (theoretical value 30+3%) in the δ¹⁸O value of the p-hydroxy groups; the first experimental data fully corroborate the predictions (see table below). Accordingly, the invention provides a method for the detection of the origin of tyrosine with respect to the feeding of animal meals to ruminants.

Other and further aspects of the present invention will become more readily apparent to those skilled in the art form the following detailed description of preferred embodiments when taken in conjunction with the drawings.

### BRIEF DESCRIPTION OF THE DRAWINGS

The invention will now be illustrated in more detail by means of preferred embodiments, with reference to the accompanying drawings.

FIG. 1 shows Scheme 1 illustrating the oxygen isotope correlations of natural substances to primary oxygen sources and equilibrium isotope effects (EIE) or kinetic isotope effects (KIE) linked to the incorporation of O. The numbers give the relevant δ¹⁸O values or their ranges in [%]_{V-SMOW}.

FIG. 2 shows Scheme 2 illustrating the biosynthesis of phenylalanine and tyrosine in plants and microorganisms. The O atom in tyrosine here comes only from (leaf) water; it comes directly from the sugar erythrose, whose O atoms undergo an enrichment of around about 30% compared to H₂O because of EIE. Phenylalanine is essential for animals; from it they make tyrosine by incorporation of O from atmospheric O₂ (δ¹⁸O=+23.5 [%]_{V-SMOW}). In doing so, the O atom experiences a depletion of around about 20%, due to KIE. PEP=phosphoenol pyruvate.

FIG. 3 shows Scheme 3 illustrating the conversion of tyrosine to 3-(p-methoxyphenyl)-1-chloropropane for the ¹⁸O analysis of the p (para) position. Me₂SO₄=dimethyl sulfate; THF=tetrahydrofuran; Ph₂P=triphenylphosphine, EtOH=ethanol.

FIG. 4 shows Scheme 4 illustrating the origin of the δ¹⁸O values of phenolic OH groups and the relative ²H pattern in tyrosine of plant and animal origin.

### DETAILED DESCRIPTION OF PREFERRED EMBODIMENTS OF THE INVENTION

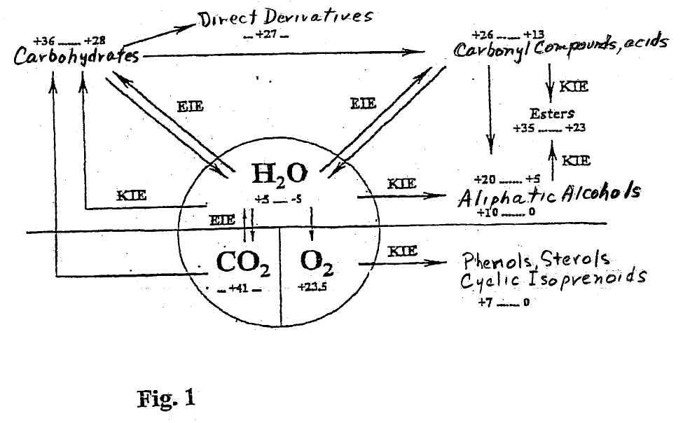

As already noted, the use of all the previously mentioned tests for origin assignments is almost exclusively based on determinations of the δ¹⁸O value of water or organic substances isotopically correlated with water. With one exception these involve determinations of global values combined with comparison of the corresponding authentic reference substances. In contrast, this invention is based on the inventors' recognition of biosynthetic relationships, interpretations and predictions of oxygen isotope contents and isotope patterns of all conceivable natural substances (and their synthetic analogs) for which the (bio)synthesis is known. In the case of hydrogen isotope patterns, this concerns above all aromatic compounds. Accordingly, based on the isotope contents of the primary sources of the oxygen in organic compounds (CO₂, H₂O, O₂), the mechanism of their (bio)syntheses and the related known and expected isotope effects (see scheme according to Schmidt et al., noted above), it is possible today to predict the δ¹⁸O of any natural substance; all of the currently available experimental data corroborate the corresponding predictions. Conversely, from the global δ¹⁸O value of a natural substance with many oxygen atoms, its δ¹⁸O values and thereby the origin of the organic compound can be derived on the basis of correlations to the leaf water of the plants that were found by the inventors (see FIG. 1, Scheme 1).

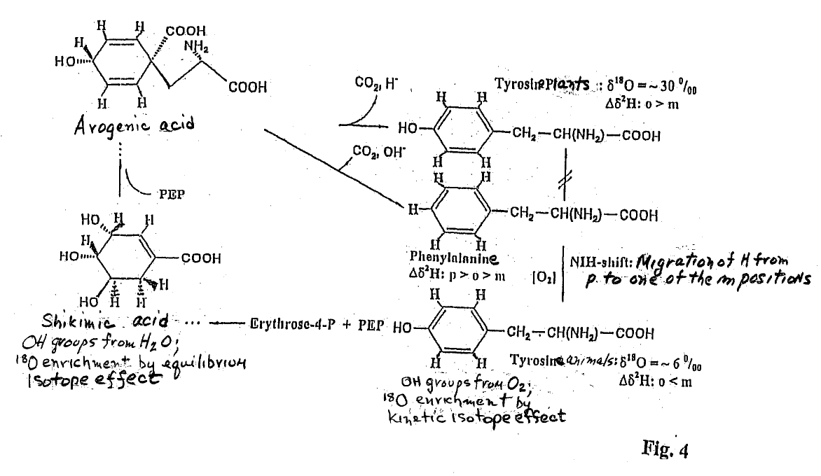

With regard to the ²H value, systematic evaluations of accessible ²H NMR data for natural substances from the group of the phenyl propane in a similar connection lead to the finding that here there is a general systematization of the relative deuterium frequency in the aromatic ring that derives from the biosynthesis mainly with the frequency sequence p>o>m, (p=para; o=ortho; m=meta) while in the case of synthetic analogs a statistical deuterium distribution is found as expected, see FIG. 4 (Scheme 4).

In addition, natural aromatic compounds, in connection with hydroxylation in p position, must experience a change of the relative positional ²H frequency because of the reaction mechanism (NIH shift, migration of the H atom [enriched in this connection] from the substituted position to the adjacent position).

The object of the invention accordingly is to provide new methods in order to

a) predict and determine the origin and authenticity of flavorings, foods and ingredients via the δ¹⁸O value without first producing data banks of corresponding comparison substances, and

b) predict the suitability of test substances for reliable origin determination or other characterization of foods, flavorings or natural drugs, as well as, with respect to other questions, for example from the field of nutrition.

The foregoing is accomplished by the present invention which constitutes a method for determining or detecting the origin of substances with biological, biochemical and nutritional physiological relevance and distinguishing them from synthetic analogs using their relative positional oxygen or hydrogen isotope ratios (δ¹⁸O value and δ²H value) comprising the steps of determining—based on the fact that for organic compounds there are at least two different primary or secondary sources for these elements, each with different isotope values, and that the incorporation of these elements is accompanied by a different, but characteristic isotope effect—the positional δ¹⁸O values or δ²H values individually in the substances to be tested, comparing them with the values expected from the biosynthesis, and detecting where a correspondence or deviation makes it possible to determine the authenticity or origin or adulteration of the substance. In a further refinement of the method, the step is included of selecting from the average value of all of the positional isotope values that occur in the substance to be tested, one of the said isotopes, measuring said isotope, comparing with the average δ¹⁸O value expected from the correlation to the starting substances, and making a determination concerning the authenticity or origin or adulteration of the substance. The method of the present invention also includes the step of determining, where in biological material the origin of the oxygen from leaf water (δ¹⁸O value +5 to −5 [%]_{V-SMOW}, 2-8 [%]_{V-SMOW} over that of the relevant ground water) or atmospheric O₂ (δ¹⁸O value+23.5 [%]_{V-SMOW}), the O atoms of which experience a characteristic isotope discrimination upon incorporation. The invention further contemplates the step of comparing, the positional hydrogen (δ²H) isotope value in aromatic compounds to be determined by ²H NMR with the relative ²H frequency p>o>m to be expected for natural substances arising by the shikimic acid pathway or for substances substituted in p position with OH, o>m or m>o, in each case according to biosynthesis. The method also contemplates that the substance to be tested, L-tyrosine is in the form of a metabolite or a component of a (macro)molecule or molecular aggregate. Also, the method includes the step of determining the plant or animal origin of tyrosine on the basis of the different origin of oxygen and/or hydrogen and the different isotope effects when they are incorporated into tyrosine.

The ¹⁸O isotope frequencies and patterns of natural substances and the corresponding synthetic analogs should be derived on the basis of known (bio)syntheses, their mechanisms, the reaction conditions and isotope effects. On this basis, experimental data for unknown samples undergo an interpretation of their origin and authenticity. In particular, the differentiation of L-tyrosine of plant or animal origin is initially intended as a basis for detection of the use of animal protein sources in the feeding of ruminants.

The positional determination of the δ¹⁸O values of L-tyrosine in order to detect the feeding of animal meals to ruminants, thus detection of circumvention of the corresponding prohibition, is proposed as a corresponding example. Tyrosine from purely animal biosynthesis (from phenylalanine) [theoretical δ¹⁸O value 5+2%] has to differ characteristically and unambiguously from that from plant sources (theoretical value 30+3%) in the δ¹⁸O value of the p-hydroxy groups; the first experimental data fully corroborate the predictions (see table below).

By means of this example, the invention demonstrates the possibilities of the scientific determination, interpretation and prediction of oxygen isotope ratios on the basis of the findings that are illustrated in more detail below and that give them general importance creating the corresponding causal connections in other examples (differentiation of plant or animal glycerins, natural or synthetic isoprenoid or phenylpropanoid flavorings) for practical use.

Based on a corresponding law, described for the first time by the inventors, the following connection is valid for patterns of the isotope ²H. In natural p-hydroxylated aromatic substances, the relative ²H frequency m>o is found, as the inventors first postulated and worked out from the literature data; this is a reversal of the sequence present in the starting material (p>o>m).

This relative frequency of deuterium must also be present in the case of tyrosine (and its derivatives) if they were created by hydroxylation from phenylalanine as is the case for tyrosine that was synthesized from phenylalanine (from animals). In contrast, tyrosine from plants derives directly from arogenic acid, and must preserve the original relative ²H frequency o>m. Thus, tyrosine (and derivatives) of animal origin must differ from tyrosine of plant origin not only by the δ¹⁸O value of the OH group in p position, but also by the relative frequency of ²H in o and m position. This prediction is also theoretically derived and is absolute in character.

The measurement of the δ²H values via ²H NMR takes place in principle in accordance with G. J. Martin, et al., U.S. Pat. No. 4,550,082, especially columns 5 to 11 for the computer analysis, columns 12 to 14 for an example, and columns 15 and 16 for the technical conduct of the test. These disclosures are expressly incorporated by reference into this application and invention and combinations with characteristics of the rest of the description herein are an object of this invention.

In this invention the following substances are to be understood as substances that have biological, biochemical and nutritional physiological relevance:

a. proteins,

b. metabolites, for example amino acids, shikimic acid, phenols,

c. substances and substance mixtures that are generally important and essential for the nutrition of living beings,

d. additives for foods and animal feeds such as flavorings and feed concentrate additives,

e. odor and flavor determining natural ingredients.

In particular, the term “protein” includes peptides, i.e., lower (1-7 amino acids) and higher (7-30 amino acids) oligomers of amino acids, optionally bonded to other classes of substances, such as, sugars, phenols, etc. These substances can be interpreted in the individual case as monomer building blocks, but as a rule are parts of macromolecules or molecular aggregates.

With respect to the requirements and sources of amino acids in monogastric animals and ruminants, the proteins of most living beings are constructed from 22 amino acids; only plants and microorganisms can synthesize all of them, higher animals have to absorb many of them via food, and for them the amino acids are “essential.” Ruminants can in principle cover their amino acid requirements just on the basis of inorganic nitrogen sources, since the bacteria in the rumen synthesize amino acids and proteins from these nitrogen sources; these acids and proteins become available to the ruminants through digestion of the bacteria in the intestine. However, in practice they are dependent on the supply of external sources of amino acids, especially when high protein synthesis rates are demanded of them (milk production, rapid growth). Since most feed cereals, above all maize, are very poor in some amino acids [Belitz H.-D., Grosh W. [1985]: Textbook of Food Chemistry, 2^{nd} Edition, Springer-Verlag, Berlin, pp. 9; 24; 418], other protein sources (soy, animal meal) have to be added to the feed. The amino acids that result are then available, independent of those produced by the rumen bacteria, to the ruminant directly or via bacterial proteins produced from them [Kirchgessner, M. [1997]: Animal Nutrition: Guide for Study, Evaluation, Practice, 10^{th} Edition, DLG-Verlag, Münster, Hiltrup, pp. 84-89]. When animals are fed only plants essentially all the amino acids in the ruminant protein derive from plant and/or microbial production, while they derive from mixed sources for monogastric animals (apart from pure vegetarians) and of ruminants fed with animal meal.

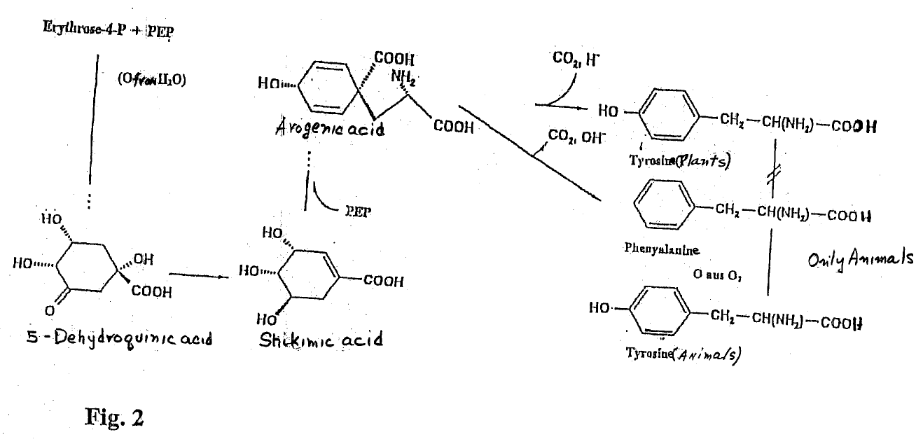

Concerning biosyntheses of L-tyrosine in plants and microorganisms or in animals, the amino acid tyrosine (Tyr) is not essential for higher animals; it is, however, produced in the animals by a single reaction step (hydroxylation) from the essential amino acid phenylalanine (Phe). Plants and microorganisms produce both amino acids from the same precursor, arogenic acid (or prephenic acid); hydroxylation of Phe to Tyr here is almost never seen [Haslam E. [1993]: Shikimic Acid, Metabolism and Metabolites, John Wiley & Sons, Chichester], see FIG. 2 (Scheme 2).

Thus, in correspondence with Scheme 2 the oxygen atom in p-position of Tyr from plant production derives from the precursor arogenic acid and thus the primary product erythrose (sugar); in the end it goes back to H₂O (with ¹⁸O enrichment due to an isotope effect). In the case of Tyr from animal synthesis it derives from atmospheric O₂, from which it is incorporated with ¹⁸O enrichment (isotope effect first postulated by the inventors) in correspondence with Scheme 2. The δ¹⁸O value in this position to be expected in accordance with Scheme 1 is +30±3% for plant Tyr (the range of variation is conditioned by the ground or leaf water; if this is known it can be “normalized” i.e., corrected), while the value for Tyr from animals produced from Phe is about +5±2%.

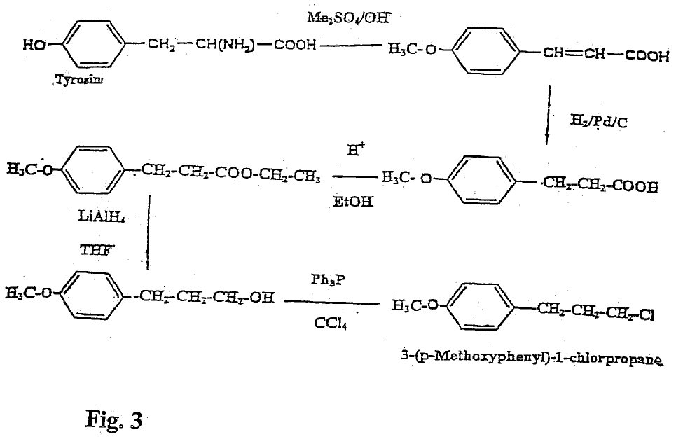

Regarding isolation of Tyr from biological material and ¹⁸O analysis in p position, Tyr is found in the amount of 6.3 wt % in the casein of milk and in the amount of 4.2 wt % in the muscle protein of cows the hydrolysis of these proteins has been described, and the isolation of the amino acid from the hydrolysate via its poor solubility is known and relatively unproblematic. For the corresponding isolation from cross-linked keratins (hair, hooves, claws) the corresponding procedures of FIG. 3 (Scheme 3) are adapted.

Before oxygen isotope analysis in p-position can be effected, the other oxygen atoms have to be eliminated, since they would have an adverse effect on the outcome. Known methods of organic chemistry were applied to develop a procedure shown in FIG. 3 (Scheme 3) that results in a satisfactory yield (50 mg Tyr is required, 10 mg 3-(p-methoxyphenyl)-1-chloropropane is obtained).

The δ¹⁸O values obtained for the p position of Tyr of various origin or of tyrosol, a natural derivative of L-tyrosine from plants, determined in this compound using known methods (see FIG. 2, Scheme 2) were expressed in [%]_{V-SMOW} (tyrosol normalized to δ¹⁸O for leaf water=0%).

TABLE

| | | | |
|---|---|---|---|
| | | Tyrosol | +30.2 |
| | | Tyr from casein Tyrosol | +23.5 |
| | | Tyr from chicken feathers | +18.9 |
| Tyr, commercial (Aldrich) | +31.5 | Tyr from human hair | +17.8 |
| | | Vanillin (as a model of a | +5.8 |
| | | p-hydroxylated compound) | |

The sources for the commercial Tyr were not known, but it is to be assumed that it was isolated from plant protein. The sample of casein completely corresponds to the expectations for Tyr that largely derives from plant and/or microbial biosynthesis and partial synthesis in the animal. The fraction of “plant tyrosine” is 70%. In contrast, the samples of animals fed with a mixed diet are in correspondence with origin from plant and animal sources (including the production in the animal from Phe); from the given expected values the fraction of plant Tyr for human hair is calculated to be 47%.

The result proves, as a matter of example, the possibility of predicting and/or determining the positional and global oxygen isotope frequencies from the mechanisms of possible syntheses. In this case, this results from the possibility of detecting a (prohibited) feeding with animal meal by means of a milk sample; beyond that, it will be possible to provide evidence of such feeding retroactively by testing on meat, hair or horns.

Although the invention has been shown and described in terms of specific preferred embodiments, changes and modifications will be evident to those of skill in the art from the disclosure and teachings herein. Such changes and modifications are deemed to fall within the purview of the inventions claims.

What is claimed is:

1. A method for determining the origin of substances with biological, biochemical and nutritional physiological relevance and distinguishing them from synthetic analogs using their relative positional oxygen or hydrogen isotope ratios (δ¹⁸O value and δ²H value) comprising the steps of determining—based on the fact that for organic compounds there are at least two different primary or secondary sources for these elements, each with different isotope values, and that the incorporation of these elements is accompanied by a different, but characteristic isotope effect—the positional δ¹⁸O values or δ²H values, individually in the substances to be tested, comparing them with the values expected from the biosynthesis, where a correspondence or deviation makes it possible to detect the authenticity or origin or adulteration of the substance, and detecting the authenticity or origin or adulteration of the substance.

2. A method as in claim 1, comprising the further step of measuring one isotope in respect of the global value, namely, the average value of all of the positional isotope values that occur in the substance to be tested, compared with the average δ¹⁸ O value expected from the correlation to the starting substances, and detecting the authenticity or origin or adulteration of the substance responsive to the comparison.

3. A method as in claim 1, including the step of determining in biological material the origin of the oxygen from leaf water (δ¹⁸O value +5 to −5 [%]_{V-SMOW}, 2-8 [%]_{V-SMOW} over that of the relevant ground water) or atmospheric O₂ (δ¹⁸O value+23.5 [%]_{V-SMOW}), the O atoms of which experience a characteristic isotope discrimination upon incorporation.

4. A method as in claim 1, including the steps of determining by ²H NMR the positional hydrogen (δ²H) isotope value in aromatic compounds, and comparing with the relative ²H frequency p>o>m to be expected for natural substances arising by the shikimic acid pathway or for substances substituted in p position with OH, o>m or m>o, in each case according to biosynthesis.

5. A method as in claim 1 including the step of testing L-tyrosine in the form of a metabolite or a component of a (macro)molecule or molecular aggregate.

6. A method as in claim 1 including the step of detecting the plant or animal origin of tyrosine on the basis of the different origin of oxygen and/or hydrogen and the different isotope effects as incorporated into tyrosine.
